(19) United States
(12) Patent Application Publication
(10) Pub. No.: US 2025/0242141 A1
(43) Pub. Date: Jul. 31, 2025

(54) Graduated Dose Dispenser And Applicator

(71) Applicant: Davies Pharmaceuticals LLC, Crown Point, IN (US)

(72) Inventors: Robb Davies, Crown Point, IN (US); Brent Page, Crown Point, IN (US)

(21) Appl. No.: 18/422,214

(22) Filed: Jan. 25, 2024

(51) Int. Cl. A61M 37/00

(52) U.S. Cl. CPC A61M 37/00; A61M 2207/10

(57) ABSTRACT

A graduated dose dispenser and applicator for topical medications wherein medications may be dispensed in an accurate and safe manner. The graduated dose dispenser and applicator utilizes a body, plunger, screw rod, pin, knob, channel, plunger lip, detent, alignment ribs, and rounded applicator tip to accurately dispense and apply topical medications.

### BACKGROUND OF THE INVENTION

The present subject matter is the first of the kind delivery container that aids in soothing pleasant transdermal medication delivery. The topical medication delivery device allows for exact dosage amounts with each dispense of medication, which does not exist in any other prescription container of the gel and gel-cream medications.

In the prior art, medication is typically given in a patch, pill, injectable, or capsule form. The present subject matter is the first of its kind that aids in soothing pleasant transdermal delivery in an accurate manner.

The present subject matter resolves many issues currently caused by oral, injectable, and patch medications. Some of this issues include irritation of skin, anxiety from the use of needles, and also will allow for discrete method application of medication in public settings. The present subject matter aids in delivering medication that is applied transdermally and can directly go to the nervous system and body instead of having to use the GI tract and get certain dose percentages lost in its application.

### SUMMARY OF THE INVENTION

The present subject matter comprises of the following parts:
5—Cap
10—Body
15—Plunger
20—Screw Rod
25, 26—Pins
30—Knob
35—Channel
40—Plunger Lip
45, 46—Detent
50—Alignment Ribs
55—22-400 Plastic Bottle Thread
60—Round Tip

The cap, body, and screw rod/knob assembly are comprised of polyethylene terephthalate or other suitable pharmaceutical grade rigid plastic. The plunger is comprised of polypropylene or other suitable pharmaceutical grade pliable plastic.

The present subject matter operates as follows:

The plunger 15 is threaded onto the screw rod 20. The screw rod 20 assembly is then inserted into the body 10. Once assembled, the unit cannot be separated.

The cap 5 and body 10 are assembled with a 22-400 plastic bottle thread 55 for a complete and tight seal.

The channel 35 of the body 10 feature two detents 45, 46 which are located 180 degrees apart. One pin 25 is enlarged to catch the detent 45 when twisting knob 30.

The present subject matter is designed to dispense an exact amount of product per one-half turn of knob 30.

The round tip 60 of the body 10 is used as the product applicator.

### BRIEF DESCRIPTION OF DRAWINGS

FIG. 1 shows a perspective disassembled view of the graduated dose dispenser and applicator.

FIG. 2 shows a side cutaway view of an assembled graduated dose dispenser and applicator.

FIG. 3 shows a 5:1 magnified cutaway view of the graduated dose dispenser and applicator.

FIG. 4 shows a 4:1 magnified cutaway view of the graduated dose dispenser and applicator.

FIG. 5 shows a 2:1 magnified top cutaway view of the B-B cross section.

FIG. 6 shows a 6:1 magnified cutaway view of the outer perimeter of the B-B cross section.

FIG. 7 shows a cutaway view of the A-A cross section which is a magnified of the tip of FIG. 8.

FIG. 8 shows a side cutaway view of the graduated dose dispenser and applicator.

FIG. 9 shows a side view of the assembled graduated dose dispenser and applicator which is 125.66 mm in height.

### DETAILED DESCRIPTION OF THE INVENTION

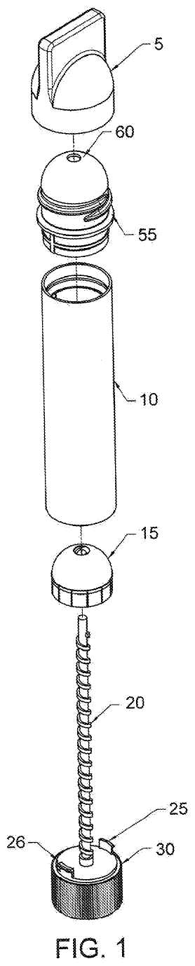

FIG. 1 shows a perspective disassembled view of the graduated dose dispenser and applicator wherein cap 5 is removably coupled with round tip 60. Body 10 slides vertically over plunger 15, screw rod 20, and is affixed to knob 30.

FIG. 2 shows a side cutaway view of an assembled graduated dose dispenser and applicator wherein plunger lip 40 presses against the interior surface of body 10 to help accurately dispense a topical medication. The channel 35 features two detents 45, 46 which are located 180 degrees apart wherein pin 25 is enlarged to catch the detent 45 when twisting knob 30.

FIG. 3 shows a 5:1 magnified cutaway view of the graduated dose dispenser and applicator wherein plunger lip 40 presses against the interior surface of body 10 to help accurately dispense a topical medication.

FIG. 4 shows a 4:1 magnified cutaway view of the graduated dose dispenser and applicator wherein detent 45 is located 180 degrees apart from detent 46.

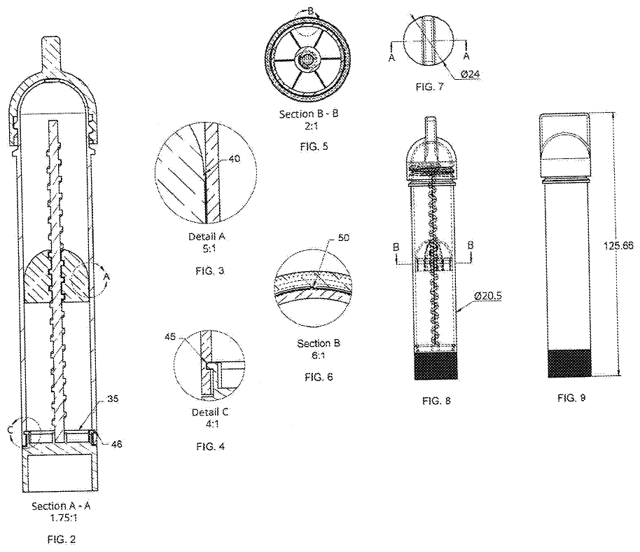

FIG. 5 shows a 2:1 magnified top cutaway view of the B-B cross section wherein alignment ribs 50 rotate to accurately dispense a topical medication.

FIG. 6 shows a 6:1 magnified cutaway view of the outer perimeter of the B-B cross section wherein alignment ribs 50 rotate to accurately dispense a topical medication.

FIG. 7 shows a cutaway view of the A-A cross section.

FIG. 8 shows a side cutaway view of the graduated dose dispenser and applicator.

FIG. 9 shows a side view of the assembled graduated dose dispenser and applicator.

1. A graduated dose dispenser and applicator comprising of:
a) cap;
b) body;
c) plunger;
d) screw rod;
e) pins;
f) knob;
g) channel;
h) plunger lip;
i) detents;
j) alignment ribs;
k) 22-400 plastic bottle thread; and
l) round tip.

2. The graduated dose dispenser and applicator as in claim 1, wherein the graduated dose dispenser and applicator is designed to dispense an exact amount of product per one-half turn of knob.

3. The graduated dose dispenser and applicator as in claim 1, wherein two detents are located 180 degrees apart so as to catch pin 25 when twisting the knob.

4. The graduated dose dispenser and applicator as in claim 1, wherein the body slides vertically over the plunger, screw rod, and affixed to the knob.

5. The graduated dose dispenser and applicator as in claim 1, wherein the plunger lip presses against the interior surface of the body to accurately dispense a topical medication.

6. The graduated dose dispenser and applicator as in claim 1, wherein alignment ribs rotate to accurately dispense a topical medication.

7. A method of constructing a graduated dose dispenser and applicator comprising of:
a) cap;
b) body;
c) plunger;
d) screw rod;
e) pins;
f) knob;
g) channel;
h) plunger lip;
i) detents;
j) alignment ribs;
k) 22-400 plastic bottle thread; and
l) round tip.

8. The method of constructing the graduated dose dispenser and applicator as in claim 7, wherein the graduated dose dispenser and applicator is designed to dispense an exact amount of product per one-half turn of knob.

9. The method of constructing the graduated dose dispenser and applicator as in claim 7, wherein two detents are located 180 degrees apart so as to catch pin 25 when twisting the knob.

10. The method of constructing the graduated dose dispenser and applicator as in claim 7, wherein the body slides vertically over the plunger, screw rod, and affixed to the knob.

11. The method of constructing the graduated dose dispenser and applicator as in claim 7, wherein the plunger lip presses against the interior surface of the body to accurately dispense a topical medication.

12. The method of constructing the graduated dose dispenser and applicator as in claim 7, wherein alignment ribs rotate to accurately dispense a topical medication.
